(19) United States
(12) Patent Application Publication
(10) Pub. No.: US 2008/0280355 A1
(43) Pub. Date: Nov. 13, 2008

(54) Control of Gene Expression with the Use of a Transcription Attenuator

(76) Inventors: Eduardo Santero Santurino, Sevilla (ES); Angel Cebolla Ramirez, Sevilla (ES); Jose Luis Royo Sanchez-Palencia, Sevilla (ES)

Correspondence Address: ARENT FOX LLP, 1050 CONNECTICUT AVENUE, N.W., SUITE 400, WASHINGTON, DC, 20036, US

(21) Appl. No.: 11/667,007

(22) PCT Filed: Oct. 11, 2005
(86) PCT No.: PCT/ES2005/000541

§ 371 Date: Jun. 5, 2008

(30) Foreign Application Priority Data
Nov. 4, 2004 (ES) P2004026650

(51) Int. Cl. C12N 15/74

(52) U.S. Cl. 435/320.1

(57) ABSTRACT

This invention relates to a system for the expression of heterologous genes, comprising an attenuator element which inhibits the elongation of the transcription of the heterologous genes, the expression of which is to be controlled, and two regulating modules which control the expression of the attenuator element. The invention also relates to the use of said expression system for the amplification of the expression of recombinant proteins, RNAs or apolipoproteins in bacteria. The invention further relates to vectors containing said expression system.

### TECHNICAL FIELD

The present invention falls within the field of genetic engineering. More specifically, it relates to the manipulation of gene expression in heterologous bacterial expression systems, in which a reduction of basal transcription levels is achieved by using a transcription attenuation system, maintaining the original maximum levels of transcription.

### STATUS OF PREVIOUS TECHNIQUES

The production of recombinant proteins by genetically modified bacteria employs strong promoters that can be suppressed as well as weak promoters that can be activated by transcription regulators.

Often, the expression of heterologous proteins in bacteria can negatively affect the growth of host bacteria. Thus, expression systems are usually kept “shut down” until the bacterial cultures reach the appropriate density, and it is then that the production of the protein of interest is induced. The problem lies in the fact that the so-called simple systems, even under basal conditions, generate a certain amount of heterologous proteins, which may lead to the selection of clones within the culture that do not express the protein of interest, if this is toxic to bacterial metabolism.

The majority of prokaryotic expression systems use multicopy plasmids that incorporate strong transcription initiation signals that can be recognized by bacterial or viral RNA polymerases. Natural systems of regulation usually include additional control circuits that regulate levels of expression in time and space. The use of additional control steps in expression vectors may help to coordinate the expression of different proteins or to improve the yield of the heterologous recombinant proteins obtained (Chen W. et al, Gene. 1993; 130(1): 15-22; Cebolla A. et al, Nucleic Acids Res. 2001; 29(3): 759-66). Diverse systems of regulation alternative to initiation control have been described. These include RNA stability regulation (lost I. et al, J. 1995; 14(13): 3252-62; Carrier T. A. et al, Biotechnol. Prog. 1999; 15(1); 58-64), translation efficiency regulation (Hui A. et al, EMBO J. 1984; 3(3): 623-9), and the regulation of protein stability proper on the part of the proteins produced (Alexander D. M. et al, Protein Expr. Purif. 1992; 3(3): 204-11). Although these additional levels of regulation may provide advantages to silence protein expression under non-induced conditions, few among them have been used in expression systems.

Different strategies have been developed to diminish the basal transcription levels of the different expression systems. Some authors have generated mutations in the area of the promoter so as to reduce its activity. The problem with this strategy is that when the moment comes to activate the system, its maximum production capacity is equally restricted.

As an alternative formula to reducing basal levels of expression, the reduction of the dosage of heterologous genes expressed has been sought by using low-copy expression vectors or integrating the said genes into the bacterial chromosome. These strategies also bring about a reduction in the performance of the system and frequently make the addition of steps to the production processes obligatory.

Other strategies have been based on the use of operators in the promoter regions in combination with their respective repressors. Apart from repressors it is also possible to coexpress different proteins that decrease basal transcription levels through diverse molecular mechanisms.

Among the varied mechanisms for controlling gene expression, transcription attenuation has always been considered as a very sophisticated and useful bacterial strategy. Many means of attenuation have been studied in a broad range of microorganisms such as Escherichia coli, Klebsiella pneumoniae, Salmonella typhimurium or Bacillus subtilis (Rutberg B. et al, Mol. Microbiol. 1997; 23(3): 413-21). The principal characteristic of attenuation mechanisms is that they prevent the non-specific elongation produced by spurious bonding between bacterial polymerase RNA and the promoter.

ES 2.167.161 describes an expression circuit based on different regulating elements of Pseudomonas putida. In this system, the nahR/Pₛₐₗ-xylS2 fusion module is inserted into the bacterial chromosome by means of a mini-Tn5 delivery system. When salicylate is present in the culture medium, NahR activates transcription from Pₛₐₗ, thus producing XylS2. At the same time, the salicylate also activates the regulating function of XylS2, synergetically amplifying transcription from the Pm promoter. In the absence of salicylate, the basal levels of expression are minimal due to the low concentration of XylS2 and its inactive status. However, this type of cascaded regulatory circuit cannot prevent residual levels of transcription initiation signals from the Pm promoter, particularly when found in a high-copy plasmid, since—even in the absence of its XylS2 regulator—bacterial RNA polymerase is capable of sporadically initiating transcription.

### AN EXPLANATION OF THE INVENTION

As has been previously indicated, the problem lies in the fact that the so-called simple systems generate a certain amount of proteins even under non-induction conditions, which may lead to the domination of the culture by clones that may have lost the capacity to express the protein of interest. The inventors have designed a system that exercises its control over transcription elongation, and thus may be superimposed upon the different levels of expression based on the start of the transcription described up to now, in such a way as to increase the efficiency of cloning in heterologous expression systems and the stability of the strains containing the resulting gene constructions.

In keeping with the first aspect of the present invention, an expression system for heterologous genes is provided that includes a transcription-promoting sequence, an attenuating element that inhibits transcription elongation, and at least one heterologous gene, the expression of which is to be controlled.

As per a preferred method of execution, the attenuation system may be counteracted or annulled in a controlled manner through the expression of a specific antiterminating protein incorporated into the system, the activity of which may be induced by an enabling molecule acting directly or indirectly on the said protein.

As per an even more preferred method of execution, the system includes the gene that encodes the antiterminating protein. In accordance with a more preferred method of execution, the promoter that initiates the transcription of heterologous genes is activated by the same molecule that activates the expression of the antiterminating protein.

In a particularly preferred execution of the first aspect of the present invention, the gene expression system includes a transcription promoting sequence, the K. pneumoniae nasF operon attenuating sequence, the K. pneumoniae nasR gene sequence, a system of heterologous operation to control the expression of the K. pneumoniae nasR gene, and one or several heterologous genes, the expression of which is to be controlled.

Of particular preference is the gene expression system in accordance with what was previously described, in which the heterologous expression system controlling the K. pneumoniae nasR gene expression is the cascade expression system nahR/Pₛₐₗ-xylS2.

As per a second aspect of the present invention, the use of the expression system previously described is provided for the amplification of the expression of recombinant proteins, RNA or apoliproteins in bacteria.

As per a third aspect of the present invention, it provides a method for improving the expressive capacity of heterologous genes in bacteria, characterized in that it includes the following stages:

(i) reduction of the basal levels of expression of the gene or genes, the expression of which is to be controlled, through a system of transcription attenuation:

(ii) activation of a heterologous expression system that expresses a protein that provokes the antitermination of the attenuation system, at the same time that it activates the transcription promoter of the gene or genes, the expression of which is to be controlled.

As per a fourth aspect of the present invention, it provides the use of a system of attenuation to improve the expressive capacity of an expression system through the reduction of the basal levels of expression of the heterologous protein.

In another, even more preferred execution, the attenuation system may be antiterminated through a protein, the activity of which may be induced by an enabling molecule either acting directly on the said protein or on the intracellular level of the said protein.

It is particularly preferred that the attenuation system contain the attenuating sequence of the K. pneumoniae nasF operon. And it is much more highly preferred that the attenuation system contain, in addition, the sequence of the Klebsiella nasR gene under the control of a heterologous expression system, in order to control the attenuating activity of the Klebsiella nasF operon.

As per a fifth aspect of the present invention, it provides expression vectors of heterologous genes that contain a promoter transcription sequence, a transcription attenuating element, and a heterologous gene or genes, the expression of which is to be controlled.

As per a preferred execution of this fifth aspect of the invention, the vectors, moreover, include a gene that encodes an antiterminating protein that can in turn prevent elongation inhibition. More preferably, the vectors, in addition, include an expression system that induces the production of the said antiterminating protein, with particular preference given to those vectors where the attenuating sequence is the K. pneumoniae nasF operon sequence. Even more preferred are those vectors in which the gene encoding the antiterminating protein is the Klebsiella nasR gene. The most highly preferred vectors are those in which the expression system that induces the production of the said antiterminating protein is the nahR/Pₛₐₗ-xylS2 cascade system.

In one practical execution of the present invention, an attenuating element is placed between a transcription-promoting sequence and one or several heterologous genes, the expression of which is to be controlled. The attenuating element is capable of prematurely interrupting transcription from the promoter and thus reducing basal levels of expression. In this way, the levels of basal expression may be decreased by more than one order of magnitude. It is possible to control the attenuating activity of this element by means of a counteracting protein and to enable transcription to continue for heterologous genes. The attenuating effect is eliminated by inducing the expression of the antiterminating protein, making a maximum activation of the promoter possible.

The insertion of the heterologous gene sequence may be done by means of restriction and ligation enzymes, or through site-specific recombination.

The present invention also makes reference to bacterial strains that contain some type of vector having the characteristics previously described.

### DEFINITIONS

Before the detailed discussion of the invention's forms of execution, some definitions of specific terms in relation with its principal aspects are provided.

The term “expression vector” as used here applies to the DNA molecule to which the DNA molecule bearing the protein encoding nucleotide sequence for the RNA or the protein of interest forms a covalent bond, facilitating the replication and transcription of the said sequence by the host cell once the vector has been transferred inside the said cell. A great variety of expression vectors for experimental purposes are known to experts in the field.

Throughout the description of the invention and its claims, the word “includes” and its variants do not aspire to exclude other technical characteristics, additives, components or steps. For experts in the field, other objects, advantages and characteristics of the invention shall derive partly from the description and partly from the practical use of the invention.

### BRIEF DESCRIPTION OF THE DRAWINGS

FIG. 1. Diagram of the constructions employed. The relevant restriction sites are indicated. Bla corresponds to the resistance gene with respect to β-lactamic antibiotics. The double loops represent the nasF attenuator. The filled-in circles represent transcription terminators, while the empty circles represent the oriV.

FIG. 2. Diagram of the different degrees of transcription represented by the system of modular expression. When neither XylS2 nor NasR are present in the cytoplasm, the nasF attenuator holds off non-specific transcription (A). When salicylate is added to the culture medium, XylS2 is activated and bonds to the Pm promoter, provoking high initiation of lacZ transcription, which is largely held back by the attenuating element (B). When nasR expression is induced, the antitermination increases the expression levels of β-galactosidase, even in the absence of nitrate (C). The system is completely induced when IPTG and salicylate are both added to the culture medium along with nitrate for NasR activation (D).

FIG. 3. (A) Comparison between the basal levels of β-galactosidase (measured in Miller Units, M.U.) produced by pMPO6_{terNaSR} and pMPO6 on a CC118 4S2 base. The grey and green bars correspond to pMPO6_{terNaSR} only in the absence and presence of nitrate, respectively. The black and red bars are equivalent to these, albeit in the presence of pMPO8. Blue and pink correspond to pMPO6_{terNaSR} in the presence of IPTG (without nitrate and with nitrate, respectively). Equivalent conditions with pMPO6 are represented in brown and white. (B) Percentage of transcription level terminating capacity (T %) with respect to the original vector, following the order described above.

FIG. 4. Miller Units (M.U.) produced by pMPO6_{terNaSR} and pMPO6 over an induction of 6 hours with 2 mM salicylate. The grey and green bars correspond to cultures containing induced pMPO6_{terNaSR} in the absence and presence of nitrate, respectively. The black and red bars represent a similar assay, but in the presence of pMPO8. Blue and pink correspond to pMPO6_{terNaSR} in the presence of IPTG (without nitrate and with nitrate). Lastly, the induced levels of pMPO6 (brown and white) represent levels without nitrate and with nitrate. The data shown here correspond to the mean of three independent experiments.

FIG. 5. Induction levels shown by CC118 4S2 pMPO6_{terNasR} with NasR supplied from pMPO24 (purple) or pMPO25 (blue), without nitrate (white) or with nitrate (lined). By way of control, the induced levels of pMPO6 are represented (brown and white, without nitrate and with nitrate). The data represent Miller Units after 6 hours of induction.

FIG. 6. Hybrid circuit design composed of the regulating modules nahR/Pₛₐₗ-xylS2; Pₛₐₗ-nasR, and its Pm-nasF target sequences. 2 mm of salicylate are required for induction. 0.2 g/l of nitrate must be added for antitermination.

The following modes of execution are provided by way of illustration. They are not meant to restrict the present invention.

### DETAILED EXPLANATION OF MODES OF EXECUTION

### Example 1

### Construction of the Expression System

The attenuating element of the K. pneumoniae nasF operon located downstream from the Pm promoter of the cascade system—a multiple cloning site for cloning genes of interest after the attenuator—and the sequence encoding nasR under the control of an induceable expression system was used as an example. A cascade system such as that of nahR/Pₛₐₗ-xylS2, which coordinates the expression of the heterologous gene promoter and the antiterminating protein, was used as a preferential system. The system underwent an improvement in its regulation capacity, decreasing basal expression 12-fold without limiting its production capacity once induced. In this manner, induction ranges of over 1,700 times were achieved.

### Plasmids and Conditions for Strain Growth

Both the plasmids and the strains employed are described in Table 1.

TABLE 1

| | Characteristics | Reference |
|---|---|---|
| Strains | | |
| E. coli DH5α | deoR, endA1, gyrA96, recA1, supE44 | Laboratory collection |
| E. coli S171-λpir | F recA, hsdR, RP4-2 (Tc::Mu) (Km::Tn7) lisogenized with λpir phage | De Lorenzo et al, Gene 1993, |
| | | 130: 41-6 |
| Klebsiella pneumoniae Ma51 | Wild Klebsiella strain | Laboratory collection |
| E. coli CC118 4S2 | phoA20 thi-1 rspE rpoB argE (Am) recA1 with a Km mini-Tn5 containing | Cebolla et al. Nucleic Acids |
| | the nahR/Pₛₐₗ-xylS2 fusion | Res 2001, 29: 759-66 |
| Plasmids | | |
| pCAS | Ap^{R}, expression vector with rrnBT1-Pm::multiple cloning site (MCS) | Active motif |
| pCAS_{terNasR} | Ap^{R}, expression vector with rrnBT1T2-Pm-nasF -MCS fusion, and the | This study |
| | origin of ColE1 replication | |
| pCNB4-S2 | Ap^{R}, Km^{R}, mini-Tn5 with the nahR/Pₛₐₗ-xylS2 fusion between sites I and O | Cebolla et al. Nucleic Acids |
| | | Res 2001, 29: 759-66 |
| pIZI016 | Gm^{R}, expression vector derived from pBBR with lacI^{q} and P_{tac}, broad | Moreno Ruiz et al, J Bacterial |
| | spectrum replication origin of host | 2003, 185: 2026-30 |
| pMPO6 | Ap^{R}, pCAS with rrnBT1-Pm- galK′::′lacZ fusion. | This study |
| pMPO6_{terNasR} | Ap^{R}, pCAS with rrnBT1-Pm- nasF attenuator -galK′::′lacZ fusion. | This study |
| pMPO7 | Ap^{R}, Bluescript with nasR cloned in EcoRV. | This study |
| pMPO8 | Gm^{R}, Plasmid derived from pIZ1016 with nasR cloned under P_{tac} control | This study |
| pMPO9 | Ap^{R}, pCAS with nasR under Pm promoter control | This study |
| pMPO10 | Ap^{R}, pCAS_{terNasR} with nasR under Pm control and with the nasF attenuator | This study |
| pMPO24 | Gm^{R}, Plasmid derived from pMPO8 with nasR cloned under Pₛₐₗ. | This study |
| pMPO25 | Gm^{R}, Plasmid derived from pMPO8 with Pₛₐₗ-nasF - nasR fusion. | This study |
| pUC19 | Ap^{R}, cloning vector with a ColB1 replication origin. | New England Biolabs |

The LB medium contained 10 g/l of tryptone, 5 g/l of NaCl and 5 g/l of yeast extract. When necessary, the LB medium was supplemented with 0.2 g/l of sodium nitrate to induce NasR-dependent antitermination. Ampicillin was used at 7.5 μg/l. The cultures were incubated at 37° C. in aerobic conditions, agitated at 150 rpm, and incubated at 30° C. after adding the inductor.

### DNA Isolation

The isolation of genomic DNA from K. pneumoniae, also known as Klebsiella oxytoca M5a1, was carried out according to the method previously described by Silberstein and Cohen (J. Bacteriol. 1987; 169: 3131-3137), with some variations. Briefly summarized, the cells from 5 ml of a culture saturated with Klebsiella were collected by centrifugation and stored frozen at −20° C. until subsequent use. The cells were thawed and resuspended in 0.4 ml lysis buffer (tris-HCl 50 mM pH 8, EDTA 10 mM, NaCl 100 mM, SDS 0.2%, RNAase 100 mg/l), and incubated at 37° C. for 30 minutes, after which 20 μl of protease-K (20 g/l) were added and they were incubated once more at 65° C. for 2 hours. The protein of the sample was extracted with phenol to eliminate nucleases and the nucleic acids were precipitated with ethanol. The genomic ADN thus obtained was resuspended in 0.5 ml of sterile milliQ water, and its concentration and purity were determined by calculating its OD₂₆₀/OD₂₈₀ ratio.

### Polymerase Chain Reaction (PCR)

The nasF attenuator was amplified by PCR using the genomic DNA of K. pneumoniae as a mold, along with the following primers: TerNasF2: 5′-GGAATTC GAG TGA ATA AAA GGT TTT GGG CAG CGC-3′ and TerNasR2: 5′-GGAATTC GCG CAA AAA AAA AGC GCC CGG CGG TGC-3′. The underlined positions correspond to EcoRI restriction sites. The PCR was carried out in a final volume of 25 μl containing 25 ng of chromosomic DNA from K. pneumoniae, 10 pg of each primer and 2.5 mM MgCl. The initial denaturing was carried out for 5 minutes at 95° C., followed by 35 cycles of amplification (95° C. for 30 seconds and 72° C. for 2 minutes), with a final extension of 5 min at 72° C. The nasR regulating gene was cloned using the following primers: NasR1F 5′-ACG GTT ATT GCT TGG CTG AAG-3′, and NasR1R: 5′-ATGAGCTC CTA CTC CTT TGG GGT TAC G-3′. The underlined nucleotides correspond to a restriction site, Sac1. The PCR contained 25 ng of chromosomic DNA from K. pneumoniae as a template, 10 pg of each primer and 2.5 mM MgCl. The initial denaturing was carried out for 5 minutes at 95° C., followed by 35 cycles of amplification (95° C. for 30 seconds, 62° C. for 30 seconds and 72° C. for 45 seconds), with a final extension of 5 min at 72° C.

### Enzymatic Determination of β-Galactosidase Activity

The pMPO6 or pMPO6_{terNasR} plasmids were transformed either alone or together with pIZ1016, pMPO8, pMPO24 or pMPO25 in CC118 4S2. These cultures were left to grow aerobically during the night in LB ampicillin and/or gentamycin where necessary. The inoculate was diluted 50 times and incubated at 37° C. When OD₆₀₀ reached the values of 0.2-0.3, the cultures were induced with salicylate (2 mM) or IPTG (1 mM) and incubated at 30° C. Where necessary, the LB medium was supplemented with 0.2 g/l of sodium nitrate. The induced and non-induced cultures were incubated at 30° C. and 150 rpm and the activities of β-galactosidase were analyzed 5 hours after the induction as previously described (Miller J, Experiments in molecular genetics. Cold Spring Harbor Laboratory Press, N.Y. 1972).

### Vector Construction

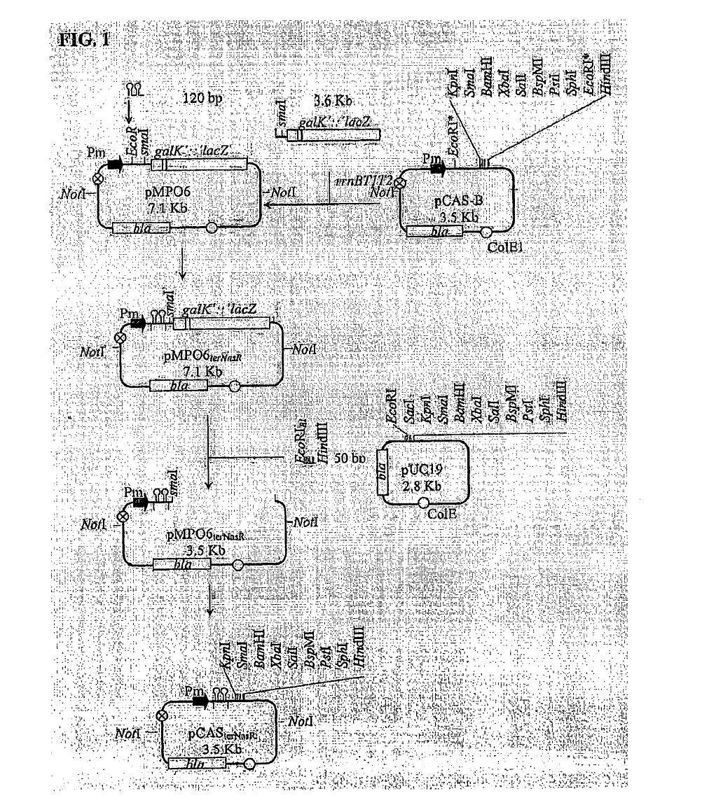

In order to evaluate the use of the nasF transcription attenuator as a filter for undesired transcription, an attempt was made to reduce the basal expression of the Pm promoter present in the cascade expression vector pCAS. The galK::lacZ fusion—which confers the best linearity between transcription level and the protein produced, due to the low stability of its encoding RNA (Cebolla et al, unpublished data)—was used to study protein expression. An EcoRI-HindIII fragment from pIC544 with this fusion (Macián F. et al, 1994; 145(1): 17-24) was inserted into the same sites as the pCAS vector, generating pMPO6 (FIG. 1). This plasmid contained a single restriction site, EcoRI, between transcription initiation (+1) and the Shine-Dalgamo (SD) sequence for the positioning of the nasF attenuator.

The 120 bp sequence corresponding to the nasF attenuator was amplified as described above, digested, and cloned in pMPO6 once linearized with EcoRI and dephosphorylated.

The primers were designed taking the sequence described by Lin et al as reference (Genebank access number AF038047). The attenuator was cloned in situ at EcoRI located upstream from the SD of the galK′::′lacZ gene. The correct orientation of the insertion was verified by means of PCR. The resulting plasmid was called pMPO6_{terNasR}.

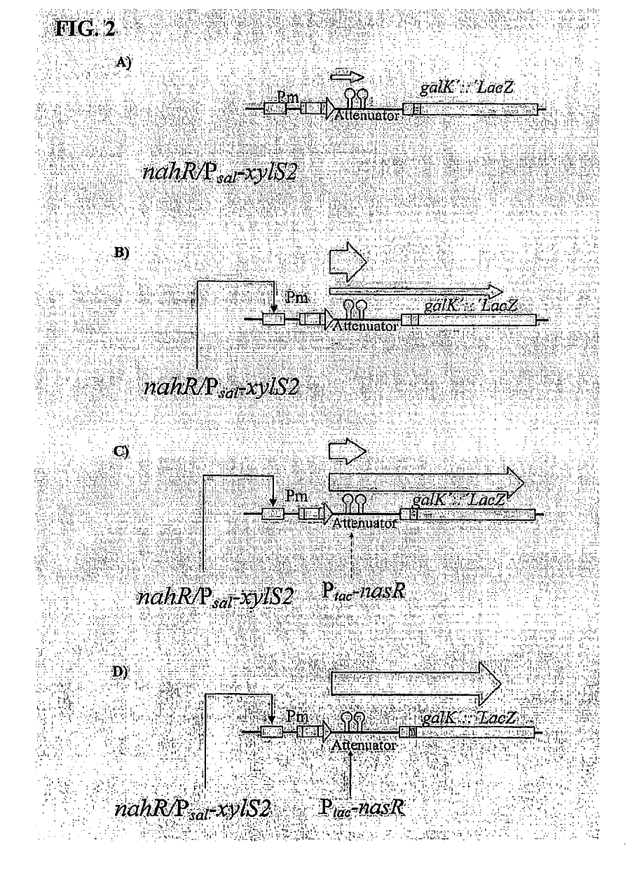

Lastly, an attempt was made to create a flexible expression vector with these properties through the insertion of a multiple cloning site downstream from the nasF attenuator. For this purpose, the pUC19 plasmid was digested with EcoRI and HindIII. The resulting 50 bp fragment containing the multiple cloning site (polylinker) was isolated and inserted into pMPO6_{terNasR} and digested with SmaI and HindIII. The resulting plasmid (pCAS_{terNasR}) made it possible to clone downstream from the Pm-nasF attenuator, as is described in FIG. 1. With these constructions, we continue to characterize the induction properties of pMPO6_{terNasR}. Some of the different configurations shown by the hybrid system are illustrated in FIG. 2. When neither active XylS2 nor NasR are present in the cytoplasm, the nasF attenuator filtered the non-specific transcription (FIG. 2A).

When salicylate is added to the culture medium, the active product XylS2 bonds to the Pm upstream from the target sequence and induces high transcription initiation. Nonetheless, the attenuator maintains control over the greater part of the potential transcriptions of lacZ (FIG. 2B). If nasR expression is induced, despite the absence of nitrate, residual antitermination increases the levels of β-galactosidase (FIG. 2C). The system is totally induced only when all the inductors are added to the culture medium. Thus, NasR activity increases, permitting antitermination and hence reaching maximum levels (FIG. 2D). These two superimposed circuits control both transcription initiation and the termination of premature elongation, making fine tuning of gene expression possible.

### The Influence of the NasF Attenuator on Basal Levels

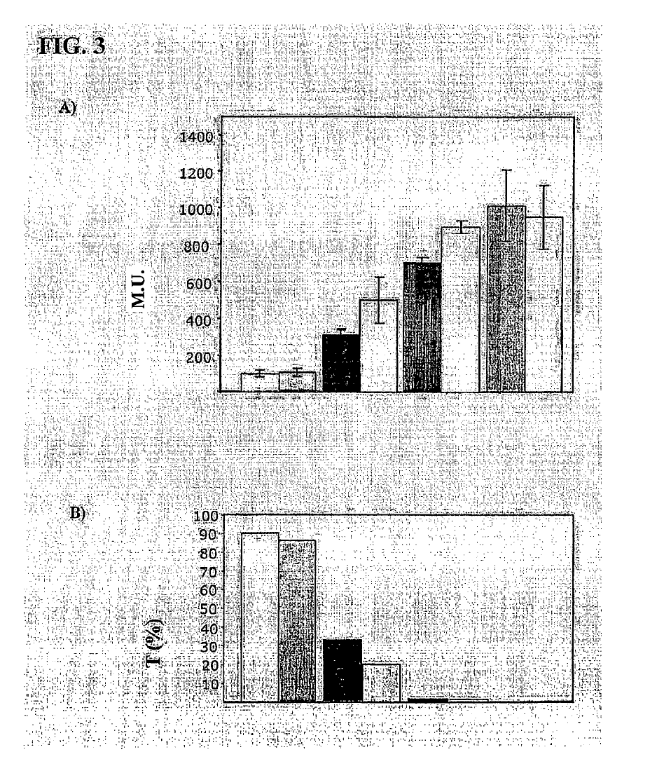

In order to quantify the effect of the nasF attenuator on basal transcription levels, pMPO6terNasR was transformed in Escherichia coli CC118 4S2. The NasR-dependent attenuator decreased the basal levels of β-galactosidase activity more than tenfold when compared with the original construction (FIGS. 2A and 3A). The strains containing pMPO6 showed an average of 1,011±196 Miller units, whereas pMPO6terNaSR showed 84±14 Miller units (n=3). This means that more than 90% of the leaked expression was filtered by the attenuator (FIG. 3B). In order to provide the system with the antiterminating protein parallel to the generation of pMPO6terNaSR, nasR (Genebank access number L27824) was amplified and the 1.3 kilobase amplicon was cloned in pBluescript (pMPO7) and afterwards digested with filled in HindIII and SacI. The resulting plasmid, pMPO8, contained the lacI^{q} repressor and expressed NasR under the control of the P_{tac} promoter. The compatibility of its replication origin with Col E2 replicons made the coexistence of both the expression vector and the plasmid modulator possible. The P_{tac} promoter enabled us to study the contribution of each parameter to the induction of lacZ. When pMPO8 was co-transformed along with pMPO6_{terNasR}, the basal levels of β-galactosidase activity increased up to 310±35 M.U., probably due to the residual antiterminating activity controlled by the expression of NasR by P_{tac} (FIG. 2B). With this configuration, the filtration capacity of the system was reduced from 90% to 35%. When enzymatic levels were analyzed in the LB 0.2 g/l nitrate culture, the basal levels increased once more up to 499±125 M.U., since the antitermination shown by NasR was activated (FIG. 2C). If nasR expression is induced by the addition of IPTG 1 mM to the culture medium, the basal levels increase once more up to 703±30 M.U. (FIG. 3A). The presence of nitrate along with IPTG was able to recover basal activity almost up to the usual levels without terminators (897±34 M.U. as against 1,011±196 M. U.), since the antitermination must have occurred with total effectiveness (FIG. 2D). We observed that the system of attenuation located in the multicopy vector expression reproduced the regulation previously described when low transcription ranges were generated.

Control Capacity Over Expression Obtained Using Combinations of NasR Expression and NahR/Pₛₐₗ-xylS2 Activation

It was set out to test whether the regulation of gene expression could be reproduced when maximum transcription ranges were attached from the Pm promoter.

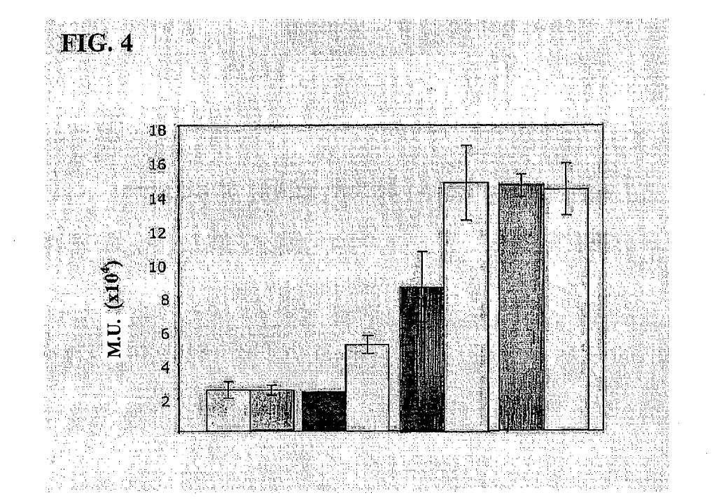

In the total absence of NasR (pMPO6terNaSR, pIZ1016), the totally induced system presented 2.45×10⁴ M.U. (292 times) (FIG. 4). With this configuration, the maximum range of induction of the system was not completely attained (16% of induced control without attenuators), since antitermination was not activated. This result also indicates the lack of an absolute termination capacity on the part of the nasF attenuator with respect to the RNA polymerase accompanied by a maximum activity of XylS2 on the Pm promoter. Nonetheless, the basal levels attained were minimum, so that this circuit may be useful particularly when it is desired to initiate the expression window at a low level. When pMPO8 was present, the basal levels of inactive NasR produced by the P_{tac} promoter were insufficient to permit the cascade expression system to reach levels superior to 2.35×10⁴ M.U. However, when NasR was activated with nitrate, the levels induced doubled (from 2.35×10⁴ to 5.11×10⁴ M.U.). The ranges of amplification under these conditions obviously depended upon the addition of nitrate. With salicylate, the levels of P-galactosidase increased 76 times, whereas, when induction was supplemented with nitrate and salicylate, amplification ranges achieved a 164-fold increase.

The residual active NasR was insufficient to permit the complete potential expression obtained when the Pm promoter was free of attenuators. When NasR production increased with the addition of 1 mM IPTG, even without nitrate, 8.57×10⁴ M.U. were obtained (60% of the level totally induced). Moreover, when nitrate, salicylate and IPTG were added, the induced levels of pMPO6terNaSR were completely achieved (1.47×10⁵ M.U.) and no differences could be detected from pMPO6 (1.45×10⁵ M.U.).

In this manner, an improvement in expression capacity from 150 to 480 times was achieved when compared with the Pm promoter without terminators (FIG. 4). When pMPO8 was present, basal levels were reduced since the cultures were deficient in nitrate. By using different conditions, a wide range of induction levels due to combinations of IPTG, salicylate and nitrate was achieved.

Escape from the P_{tac} promoter principally generated NasR. However, it prevented the complete activity of termination in the absence of any inducer. Thus, two disadvantages conditioned this circuit. First, the escape from the promoter derived from the use of P_{tac}. Second, the need for IPTG for the complete induction of the system, which may not be convenient if the production has to be increased. These two aspects could simultaneously be solved if the system were designed to include NasR expression by one of the promoters making up the cascade circuit of amplification.

Design of NasR Expression Coordinated with the Cascade System

In an attempt to increase the expression capacity of the regulating system through the use of the nasF attenuator, the expression of the nasR gene was coupled to the expression of the transcription activator by the cascade circuit. NasR expression under non-induced conditions may be minimized and co-expressed along with the other regulating elements acting on induction.

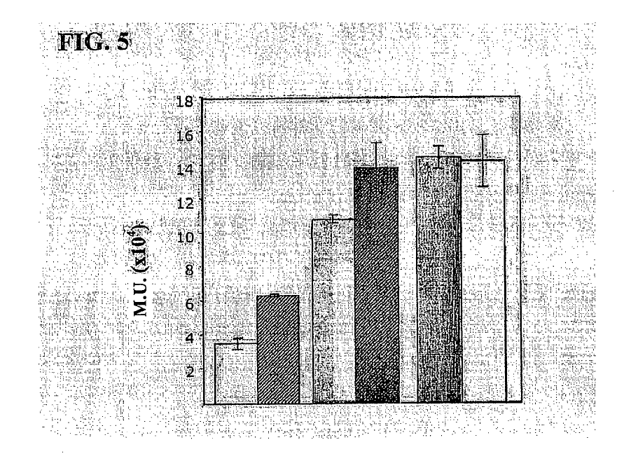

The system of cascade amplification involves two regulators: NahR and XylS2, and their respective target promoters, Pₛₐₗ and Pm. NasR was placed under the control of the promoter Pₛₐₗ in such a manner that the co-expression of XylS2 and the antitermination after the addition of salicylate were synchronized. To do this, the fragment NcoI-SalI containing IacI^{q}-P_{tac} from pMPO9 was changed and substituted by the Pₛₐₗ promoter, generating pMPO24. An alternative for pMPO24 was generated by replacing nasR with a nasR fusion attenuator, pMPO25, in the event that the basal level of nasR expression was still significant. We transformed both pMPO24 and pMPO25 along with pMPO6terNasR in CC118 4S2 and analyzed basal and activated levels. The low promoter basal activity of Pₛₐₗ (pMPO24) led to non-induced levels of 81±10 M.U., which where not distinguishable from the configuration without nasR (84±14 M.U.). As was expected, no lower basal levels were obtained from the use of pMPO25, since these were already minimal. These new configurations presented the highest induction ranges when induced with salicylate and nitrate, reaching values of 1.4×10⁵ M.U. (FIG. 5). Thus, the co-expression of nasR by Pₛₐₗ produced up to 1,711 times the induction shown by a high-copy expression vector (Table 2).

TABLE 2

| | Non-induced | | Induced | | |
|---|---|---|---|---|---|
| CC118 4S2 pMPO6_{terNasR} | −NO₃ | +NO₃ | −NO₃ | +NO₃ | Induction range |
| +pIZ1016 | 84 ± 14 | 89 ± 9 | 24,511 + 4,990 | 24,170 + 2,939 | 287 |
| +pMIPO24 | 81 ± 11 | 125 ± 49 | 108,333 ± 2,387 | 138,641 ± 14,568 | 1,711 |
| +pMPO25 | 75 ± 2 | 90 ± 20 | 35,417 ± 3253 | 63,616 ± 973 | 848 |

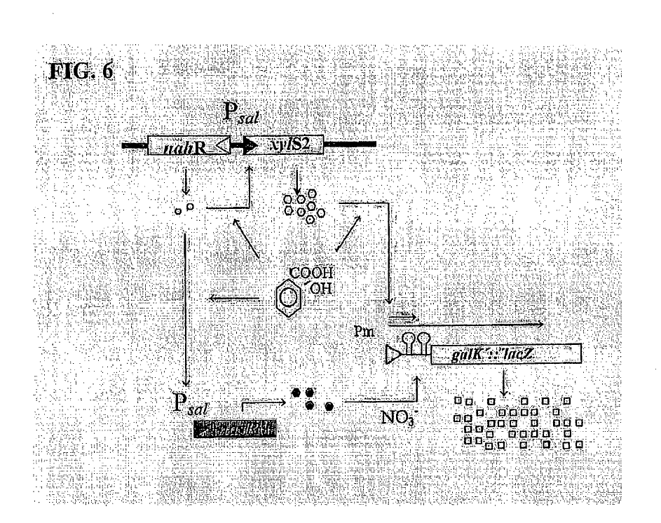

On the other hand, this circuit seemed insufficient to generate enough nasR in order to manage to completely antiterminate both attenuators located in pMPO25 and pMPO6_{terNasR}. However, this characteristic made a 848-fold induction range possible. These configurations generated the lowest basal levels while maintaining the greatest range of induction. The specific profile of expression conditioned by pMPO24 (FIG. 6) enabled us to play with the maximum activity of expression equivalent to the termination-free system of expression (FIG. 5).

1. A system of heterologous gene expression characterized in that it is made of:
(i) a sequence promoting transcription;
(ii) an attenuating element that inhibits the elongation of heterologous gene transcription; and
(iii) at least one heterologous gene, the expression of which is to be controlled.

2. A system of expression according to claim 1, characterized in that the system of attenuation may be counteracted or annulled in a controlled manner through the expression of a protein, the activity of which may be induced by one or several effector molecules.

3. A system of expression according to claim 1, characterized in that, moreover, it is comprised of a gene that encodes the antiterminating protein that can prevent inhibition of elongation.

4. A system of expression according to claim 3, characterized in that the promoter that initiates heterologous gene transcription is activated by the same molecule that activates the expression of the antiterminating protein.

5. A system of gene expression according to claim 1, characterized in that it is comprised of:
(i) a transcription promoting sequence;
(ii) the attenuating sequence of the K. pneumoniae nasF operon;
(iii) the sequence of the K. pneumoniae nasR gene;
(iv) a system of heterologous expression to control the expression of the K. pneumoniae nasR gene; and
(v) a heterologous gene or genes, the expression of which is to be controlled.

6. A system of gene expression according to claim 5, characterized in that the system of heterologous expression controlling the expression of the K. pneumoniae nasR gene is the cascade expression system nahR/Pₛₐₗ-xylS2.

7. The use of the system of expression according to claim 1 for the amplification of the expression of recombinant proteins, RNA or apoliproteins in bacteria.

8. A method for improving the capacity of heterologous gene expression in bacteria, characterized in that it is composed of the following stages:
(i) reduction of the basal expression levels of the heterologous gene or genes, the expression of which is to be controlled, through a system of attenuation;
(ii) activation of a system of heterologous expression expressed by a protein that provokes the antitermination of the system of attenuation, at the same time that it activates the transcription promoter of the heterologous gene or genes, the expression of which is to be controlled.

9. The use of a system of attenuation to improve the expression capacity of an expression system by reducing the basal expression levels of the heterologous protein.

10. Use as according to claim 9, characterized in that the system of attenuation may be antiterminated by means of a protein whose activity may be induced by an effector molecule that either acts directly on the said protein or on the intracellular level of the said protein.

11. Use as according to claim 9, characterized in that the system of attenuation contains the attenuating sequence of the K. pneumoniae nasF operon.

12. Use as according to claim 9, characterized in that the system of attenuation contains the sequence of the Klebsiella nasR gene under the control of a heterologous expression system for controlling the attenuating activity of Klebsiella nasF.

13. Expression vectors of heterologous genes characterized in that they contain a transcription-promoting sequence, a transcription-attenuating element and a heterologous gene or genes, the expression of which is to be controlled.

14. Vectors as according to claim 13, characterized in that, moreover, they are composed of a gene that encodes an antiterminating protein that can prevent inhibition of elongation.

15. Vectors as according to claim 13, characterized in that, moreover, they are composed of an expression system that induces the production of the said antiterminating protein.

16. Vectors as according to claim 13, characterized in that the attenuating sequence is that of the K. pneumoniae nasF operon.

17. Vectors as according to claim 13, characterized in that the gene encoding the antiterminating protein is the Klebsiella nasR gene.

18. Vectors as according to claim 13, characterized in that the expression system that induces the production of the said antiterminating protein is induced by salicylate.

19. Vectors as according to claim 13, characterized in that the expression system that induces the production of the said antiterminating protein is composed of nahR or a derivate of xylS.
